(19) United States
(12) Patent Application Publication
(10) Pub. No.: US 2006/0257354 A1
(43) Pub. Date: Nov. 16, 2006

(54) Ethylene copolymer waxes containing amino groups and use thereof

(75) Inventor: Andreas Fechtenkötter, Ludwigshafen (DE)

Correspondence Address: CONNOLLY BOVE LODGE & HUTZ LLP, P.O. BOX 2207, WILMINGTON, DE, 19899-2207, US

(73) Assignee: BASF Aktiengesellschaft, Ludwigshafen (DE)

(21) Appl. No.: 10/572,045

(22) PCT Filed: Sep. 24, 2004
(86) PCT No.: PCT/EP04/10720

§ 371 Date: Mar. 15, 2006

(30) Foreign Application Priority Data
Sep. 30, 2003 (DE) 10345798.4

(51) Int. Cl. C10L 1/16; A61K 9/16; A61K 31/16; A61K 31/785; C07C 237/02

(52) U.S. Cl. 424/78.27; 585/9; 524/543; 514/616; 564/152

(57) ABSTRACT

Ethylene copolymer waxes containing amino groups, and use thereof

Ethylene copolymer waxes with a molecular weight M_{w} in the range from 1000 to 20 000 g/mol, which comprise, as copolymerized comonomers, (c) from 50 to 95% by weight of ethylene, (d) from 5 to 50% by weight of at least one comonomer which has at least one alkylated or cycloalkylated amino group, each of which has bonding via a spacer to a polymerizable group, (e) from 0 to 30% by weight of other comonomers, where all % by weight data are based on the entire weight of ethylene copolymer wax.

The present invention relates to ethylene copolymer waxes with a molecular weight M_{w} in the range from 1000 to 20 000 g/mol, which comprise, as copolymerized comonomers,
(c) from 50 to 95% by weight of ethylene,
(d) from 5 to 50% by weight of at least one comonomer which has at least one alkylated or cycloalkylated amino group, each of which has bonding via a spacer to a polymerizable group,
(e) from 0 to 30% by weight of other comonomers,
   where all % by weight data are based on the entire weight of ethylene copolymer wax.

Bacterial (microorganism) colonization and spread on surfaces of substrates is undesirable in all areas of daily life where hygiene is important. Examples of products affected are textile substrates, furniture and equipment, surfaces of pipelines, packaging and containers.

EP-A 0 862 858 and EP 0 862 859 disclose that graft copolymers involving tert-butylaminoethyl (meth)acrylate as graft have microbicidal properties.

DE-A 199 52 221 discloses that polymers prepared from 8.5 ml of 2-diethylaminoethyl methacrylate and 3.5 ml of alkyl methacrylate have microbicidal properties, and DE-A 199 52 221 selects alkyl from methyl, ethyl, butyl and tert-butyl. However, the polymers disclosed have the disadvantage that their microbicidal activity is inadequate for many applications. It is also necessary to use large amounts of expensive 2-diethylaminoethyl methacrylate monomer.

DE 102 03 342 discloses that polymers substantially composed of structural units which have been derived from trialkylammoniumalkyl esters of acrylic acid or methacrylic acid, and in which two of the three alkyl groups have been selected from C₈-C₁₂-alkyl and C₈-C₁₂-alkenyl are a suitable biocidal coating for, by way of example, textile.

DE 101 27 513 discloses that nonwovens can be treated with antimicrobial polymers. Examples of the antimicrobial polymers proposed are polymers of dimethylaminopropylmethacrylamide or tert-butylaminomethacrylate or 3-aminopropyl vinyl ether.

DE 42 34 324 discloses polyethylene waxes composed of ethylene and dimethylaminoethyl acrylate with molecular weight M_{w} of 25 000 or 45 000 g/mol. The polyethylene waxes disclosed in DE 42 34 324 may be used as dispersing agents for polymeric molding compositions.

An object was to provide novel substances which are suitable for the control of microorganisms and eliminate the disadvantages of the microbicidal agents known from the prior art. A further object was to provide a process for preparing novel microbicidal substances.

For the purposes of the present invention, examples of microorganisms are bacteria and also yeasts, e.g. Candida albicans and fungi, e.g. Aspergillus niger, and also algae.

Accordingly, the ethylene copolymer waxes defined at the outset have been discovered. Inventive ethylene copolymer waxes have a molecular weight M_{w} in the range from 1000 to 20 000 g/mol, preferably from 2000 to 15 000 g/mol.

Inventive ethylene copolymer waxes conmprise, as copolymerized comonomers:
(a) from 50 to 95% by weight of ethylene, preferably from 45 to 90% by weight,
(b) from 5 to 50% by weight of at least one comonomer which has at least one alkylated or cycloalkylated amino group, each of which has bonding via a spacer to a polymerizable group, preferably from 5.5 to 45% by weight,
(c) from 0 to 30% by weight of other comonomers, preferably from 0 to 25% by weight, particularly preferably from 1 to 20% by weight,
   where all % by weight data are based on the entire weight of inventive ethylene copolymer wax.

The alkylated or cycloalkylated amino group of comonomer (b) or of comonomers (b) may have been mono- or polyalkylated or mono- or polycycloalkylated. If it is desired to copolymerize two or more comonomers (b), the various comonomers (b) may have identical or different spacers or identical or different polymerizable groups or may bear identical or different alkyl groups or cycloalkyl groups on the amino group(s). For the purposes of the present invention it is also possible for at least one comonomer (b) to have two or more alkylated or cycloalkylated amino groups, each having bonding via a spacer to a polymerizable group.

In one preferred embodiment, at least one comonomer (b) has the general formula I

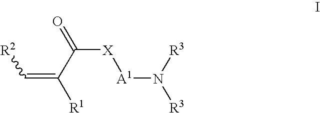

where the definition of the variables is as follows:

R¹ and R² are identical or different;

R¹ is selected from hydrogen and

unbranched and branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particularly preferably C₁-C₄-alkyl, e.g. methyl, ethyl, n-propyl, isopropyl, n-butyl, isobutyl, sec-butyl and tert-butyl, in particular methyl;

R² is selected from unbranched and branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particularly preferably C₁-C₄-alkyl, e.g. methyl, ethyl, n-propyl, isopropyl, n-butyl, isobutyl, sec-butyl and tert-butyl, in particular methyl;

and very particularly preferably hydrogen.

R³ are different or preferably identical and selected from hydrogen and branched and preferably unbranched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; preferably methyl, ethyl, n-propyl, n-butyl, n-pentyl, isopentyl, n-hexyl, n-heptyl, n-octyl, n-nonyl, n-decyl; particularly preferably C₁-C₄-alkyl, such as methyl, ethyl, n-propyl, isopropyl, n-butyl, isobutyl, sec-butyl and tert-butyl, very particularly preferably methyl;

C₃-C₁₂-cycloalkyl such as cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl,

where two radicals R³ may have bonding to one another to form a 3 to 10-membered, preferably 5- to 7-membered, ring, optionally substituted with C₁-C₄-alkyl radicals,

an N(R³)₂ group particularly preferably having been selected from

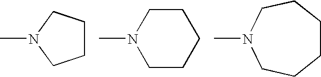

If the radicals R³ are different, one of the radicals R³ may be hydrogen.

X is selected from sulfur, N—R⁴ and in particular oxygen.

R⁴ is selected from hydrogen and unbranched and branched C₁-C₁₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particularly preferably C₁-C₄-alkyl, e.g. methyl, ethyl, n-propyl, isopropyl, n-butyl, isobutyl, sec-butyl and tert-butyl, in particular methyl;

A¹ is selected from divalent groups such as C₁-C₁₀-alkylene, e.g. —CH₂—, —CH(CH₃)—, —(CH₂)₂—, —CH₂—CH(CH₃)—, cis- and trans-CH(CH₃)—CH(CH₃)—, —(CH₂)₃—, —CH₂—CH(C₂H₅)—, —(CH₂)₄—, —(CH₂)₅—, —(CH₂)₆—, —(CH₂)₇, —(CH₂)₈—, —(CH₂)₉—, —(CH₂)₁₀—; preferably C₂-C₄-alkylene; e.g. —(CH₂)₂—, —CH₂—CH(CH₃)—, —(CH₂)₃—, —(CH₂)₄— and —CH₂—CH(C₂H₅)—, particularly preferably —(CH₂)₂—, —(CH₂)₃—, —CH₂)₄— and very particularly preferably —(CH₂)—, C₄-C₁₀-cycloalkylene, e.g.

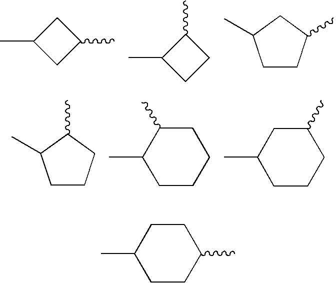

preferably

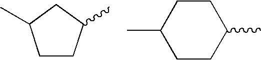

in isomerically pure form or an isomer mixture,

and

phenylene, such as ortho-phenylene, meta-phenylene and particularly preferably para-phenylene.

In one embodiment of the present invention, R¹ is hydrogen or methyl. R¹ is very particularly preferably methyl.

In one embodiment of the present invention, R¹ is hydrogen or methyl and R² is hydrogen.

In one embodiment of the present invention, R¹ is hydrogen or methyl and R²is hydrogen, and the two radicals R³ are identical, each being methyl or ethyl.

In one embodiment of the present invention, X-A¹-N(R³)₂ is O—CH₂—CH₂—N(CH₃)₂—.

In one embodiment of the present invention X-A¹-N(R³)₂ is O—CH₂—CH₂—CH₂—N(CH₃)₂—.

In one embodiment of the present invention, inventive ethylene copolymer waxes comprise no other copolymerized comonomers.

In one embodiment of the present invention inventive ethylene copolymer waxes comprise at least one other copolymerized comonomer (c). Examples of preferred other copolymerized comonomers (c) are isobutene and (meth)acrylic esters, in particular alkyl (meth)acrylates, such as C₁-C₄-alkyl (meth)acrylates.

In one embodiment of the present invention, inventive ethylene copolymer waxes have a weight-based melt flow rate (MFR) in the range from 1 to 500 g/10 min, preferably from 5 to 200 g/10 min, particularly preferably from 7 to 50 g/10 min, measured at 160° C. and with a load of 325 g to DIN 53735.

In one embodiment of the present invention, inventive ethylene copolymer waxes have a kinematic melt viscosity v in the range from 500 to 10 000 mm²/s, preferably in the range from 800 to 4000 mm²/s, measured to DIN 51562.

In one embodiment of the present invention, the melting ranges of the inventive ethylene copolymer waxes are in the range from 60 to 115° C., preferably in the range from 65 to 110° C., determined by DSC to DIN 51007.

In one embodiment of the present invention, the melting ranges of inventive ethylene copolymer wax may be wide and relate to a temperature range of from at least 5 to at most 20° C., preferably from at least 7° C. to at most 15° C.

In one embodiment of the present invention, the melting points of inventive ethylene copolymer wax are sharp and lie within a temperature range of less than 2° C., preferably less than 1° C., determined to DIN 51007.

In one embodiment of the present invention, the density of the inventive ethylene copolymer waxes is from 0.89 to 1.10 g/cm³, preferably from 0.92 to 0.94 g/cm³, determined to DIN 53479.

Inventive ethylene copolymer waxes may be alternating copolymers or block copolymers or preferably random copolymers.

Another aspect of the present invention is a process for preparing inventive ethylene copolymer waxes.

Inventive ethylene copolymer waxes may advantageously be prepared via free-radical-initiated copolymerization of ethylene with at least one comonomer which has at least one alkylated or cycloalkylated amino group, each of these groups having bonding via a spacer to a polymerizable group, and, if appropriate, from one or more other comonomers under high-pressure conditions, also termed inventive polymerization process below. By way of example, the inventive polymerization process is successfully carried out in stirred high-pressure autoclaves or in high-pressure tubular reactors or in combinations of high-pressure autoclave and high-pressure tubular reactor, these having been arranged in series. It is preferable to carry out the reaction in stirred high-pressure autoclaves. Stirred high-pressure autoclaves are known per se, and a description is found in Ullmann's Encyclopedia of Industrial Chemistry, 5^{th} edition, key words: Waxes, Vol. A 28, pp. 146 et seq., Verlag Chemie Weinheim, Basle, Cambridge, New York, Tokyo, 1996. The length/diameter ratio in these is predominantly within the ranges from 5:1 to 30:1, preferably from 10:1 to 20:1. The high-pressure tubular reactors which may likewise be used are also found in Ullmann's Encyclopedia of Industrial Chemistry, 5^{th} edition, key words: Waxes, Vol. A 28, pp. 146 et seq., Verlag Chemie Weinheim, Basle, Cambridge, New York, Tokyo, 1996.

Suitable pressure conditions for the inventive polymerization process are from 500 to 4000 bar, preferably from 1500 to 2500 bar. Conditions of this type are also termed high pressure below. The reaction temperatures are in the range from 170 to 300+ C., preferably in the range from 195 to 280° C.

The copolymerization may be carried out in the presence of at least one regulator. Examples of the regulators used are hydrogen or at least one aliphatic aldehyde or at least one aliphatic ketone of the general formula III

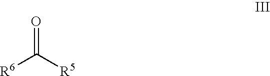

or mixtures of the same.

The radicals R⁵ and R⁶ here are identical or different and have been selected from
hydrogen;
C₁-C₆-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, particularly preferably C₁-C₄-alkyl, such as methyl, ethyl, n-propyl, isopropyl, n-butyl, isobutyl, sec-butyl, and tert-butyl;
C₃-C₁₂-cycloalkyl, such as cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl.

In one particular embodiment, the radicals R⁵ and R⁶ have covalent bonding to one another to form a 4 to 13-membered ring. For example, R⁶ and R⁵ together may be: —(CH₂)₄—, —(CH₂)₅—, —(CH₂)₆, —(CH₂)₇, —CH(CH₃)—CH₂—CH₂—CH(CH₃)— or —CH(CH₃)—CH₂—CH₂—CH₂—CH(CH₃)—.

Other examples of suitable regulators are alkylaromatic compounds, such as toluene, ethylbenzene or one or more isomers of xylene. Other examples of regulators with good suitability are paraffins, such as isododecane (2,2,4,6,6pentamethylheptane) or isooctane.

Initiators which may be used for the free-radical polymerization are the usual free-radical initiators, such as organic peroxides, oxygen or azo compounds. Mixtures of two or more free-radical initiators are also suitable.

Suitable peroxides, selected from commercially available substances, are
didecanoyl peroxide, 2,5-dimethyl-2,5-di(2-ethylhexanoylperoxy)hexane, tert-amyl peroxy-2-ethylhexanoate, dibenzoyl peroxide, tert-butyl peroxy-2-ethylhexanoate, tert-butyl peroxydiethylacetate, tert-butyl peroxydiethylisobutyrate, 1,4-di(tert-butylperoxycarbonyl)cyclohexane in the form of isomer mixture, tert-butyl perisononanoate 1,1-di(tert-butylperoxy)-3,3,5-trimethylcyclohexane, 1,1-di(tert-butylperoxy)cyclohexane, methylisobutyl ketone peroxide, tert-butylperoxy isopropyl carbonate, 2,2-di-tert-butylperoxybutane or tert-butyl peroxyacetate;
tert-butyl peroxybenzoate, di-tert-amyl peroxide, dicumyl peroxide, the isomeric di(tert-butylperoxyisopropyl)benzenes, 2,5-dimethyl-2,5-di-tert-butylperoxyhexane, tert-butyl cumyl peroxide, 2,5dimethyl-2,5-di(tert-butylperoxy)hex-3-yne, di-tert-butylperoxide, 1,3-diisopropylbenzene monohydroperoxide, cumene hydroperoxide or tert-butyl hydroperoxide; or
dimeric or trimeric ketone peroxides or cyclic peroxides of the general formula III a to III c.

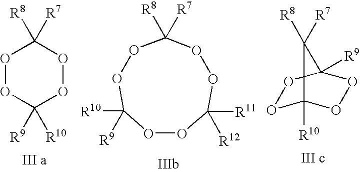

The radicals R⁷ to R¹² here are identical or different and have been selected from
C₁-C₈-alkyl, such as methyl, ethyl, n-propyl, isopropyl, n-butyl, sec-butyl, isobutyl, tert-butyl, n-pentyl, sec-pentyl, isopentyl, n-hexyl, n-heptyl, n-octyl; preferably linear C₁-C₆-alkyl, such as methyl, ethyl, n-propyl, n-butyl, n-pentyl, n-hexyl, particularly preferably linear C₁-C₄-alkyl, such as methyl, ethyl, n-propyl or n-butyl, very particular preference being given to methyl and ethyl;
C₆-C₁₄-aryl such as phenyl, 1-naphthyl, 2-naphthyl, 1-anthryl, 2-anthryl, 9-anthryl, 1-phenanthryl, 2-phenanthryl, 3-phenanthryl, 4phenanthryl and 9-phenanthryl, preferably phenyl, 1-naphthyl and 2-naphthyl, particularly preferably phenyl.

Peroxides of the general formulae III a to III c are disclosed in EP-A 0 813 550, as are processes for their preparation.

Particularly suitable peroxides are di-tert-butyl peroxide, tert-butyl peroxypivalate, tert-butyl peroxyisononanoate or dibenzoyl peroxide or mixtures of the same. An azo compound which may be mentioned by way of example is azobisisobutyronitrile (“AIBN”). The amounts added of free-radical initiators are those usual for polymerizations.

Numerous commercially available organic peroxides are treated with what are known as phlegmatizers prior to their sale in order to make their handling easier. Examples of suitable phlegmatizers are white oil or hydrocarbons, in particular isododecane. Under the conditions of high-pressure polymerization, these phlegmatizers can have the effect of regulating molecular weight. For the purposes of the present invention, the use of molecular weight regulators means the use of other molecular weight regulators in addition to the use of these phlegmatizers.

The quantitative proportion of comonomers during the addition is not usually precisely the same as the ratio of the units in the ethylene copolymer waxes used according to the invention, because comonomers which have at least one alkylated or cycloalkylated amino group, each of the groups having bonding via a spacer to a polymerizable group, are generally more easily incorporated than ethylene into ethylene copolymer waxes.

In one preferred embodiment of the present invention, the proportion of comonomer which has at least one alkylated or cycloalkylated amino group, each of the groups having bonding via a spacer to a polymerizable group, is less than 30% by weight of the entire comonomer feed, preferably less than 20% by weight.

The comonomer(s) and ethylene are usually added together or separately.

The comonomers may be compressed in a compressor to the polymerization pressure. In another embodiment of the inventive process, the comonomers are first brought to an increased pressure, for example from 150 to 400 bar, preferably from 200 to 300 bar and in particular 260 bar, with the aid of a pump, and then brought to the actual polymerization pressure by a compressor.

The inventive polymerization process may be carried out either in the absence or in the presence of solvents, and for the purposes of the present invention mineral oils, white oil and other solvents which are present during the polymerization in the reactor and have been used to phlegmatize the free-radical initiator(s) are not regarded as solvents. Examples of suitable solvents are toluene, isododecane, isomers of xylene.

The inventive polymerization process gives inventive ethylene copolymer wax, from which it is advantageously possible to remove any residual monomer still present, for example with the aid of an extruder.

In another embodiment of the present invention, the method of preparing inventive ethylene copolymer wax is that wax, obtainable via copolymerization of ethylene and of at least one comonomer having a functional group, is reacted in a polymer-analogous reaction with at least one substance which has at least one alkylated or cycloalkylated amino group and has a spacer on which there is a pendant reactive group which can react with the functional group on at least one copolymerized comonomer.

In one preferred embodiment of the present invention, wax, obtainable via copolymerization of ethylene with at least one comonomer of the general formula IV,

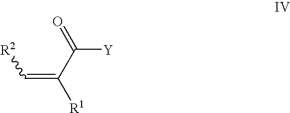

is reacted with at least one compound of the formula V,

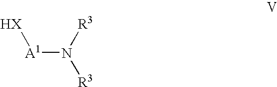

if appropriate in the presence of a catalyst, preferably in the presence of an acidic catalyst, where
Y is selected from OH and O—R¹³ and
R¹³ is C₁-C₄-alkyl, such as methyl, ethyl, n-propyl, isopropyl, n-butyl, isobutyl, sec-butyl and tert-butyl.

The copolymerization of ethylene with at least one comonomer having a functional group may be carried out by methods familiar to the person skilled in the art.

The copolymerization of ethylene with at least one compound of the general formula IV may be carried out by methods familiar to the person skilled in the art for the high-pressure polymerization of ethylene, as described in Ullmann's Encyclopedia of Industrial Chemistry, 5^{th} edition, key words: Waxes, Vol. A 28, pp. 146 et seq., Verlag Chemie Weinheim, Basle, Cambridge, New York, Tokyo, 1996.

By way of example, the polymer-analogous reaction may be carried out in a solvent.

The present invention also provides ionic ethylene copolymer waxes, obtainable via reaction of inventive ethylene copolymer waxes with Brønsted acid. The present invention also provides a process for preparing inventive ionic ethylene copolymer waxes via reaction of inventive ethylene copolymer wax with Brønsted acid.

The amino groups present in the inventive ethylene copolymer waxes are partially or completely protonated via the reaction of inventive ethylene copolymer waxes with Brønsted acid.

Examples of suitable Brønsted acids are aqueous mineral acids, such as hydrohalic acids, e.g. HCl, HBr, Hl, HF, H₂SO₄, H₃PO₄, HClO₄, HNO₃; pseudohalic acids, such as HSCN and isocyanic acid, acidic salts, such as alkali metal hydrogen sulfates, e.g. KHSO₄ and NaHSO₄, alkali metal dihydrogenphosphates, e.g. NaH₂PO₄ and KH₂PO₄, organic acids, e.g. CH₃OSO₃H, formic acid, acetic acid, oxalic acid or citric acid.

One or more inventive ethylene copolymer waxes is/are used as starting material for carrying out the inventive process for preparing ionic ethylene copolymer waxes. This or these is/are placed in a vessel, such as a flask, an autoclave or a tank, and the ethylene copolymer wax(es) and one or more Brønsted acids and, if appropriate, other substances, such as water, are heated, the sequence of addition of Brønsted acid or Brønsted acids and also if appropriate other substances being as desired. If it is desired to prepare ionic ethylene copolymer waxes at a temperature above 100° C., it is advantageous to operate under increased pressure and to select the vessel appropriately. The resultant emulsion is homogenized, for example via mechanical or pneumatic stirring or by shaking. It is advantageous to heat the material to a temperature above the melting point of the inventive ethylene copolymer wax(es). It is advantageous to heat the material to a temperature which is above the melting point of the inventive ethylene copolymer wax(es) by at least 10° C., and it is particularly advantageous to heat the material to a temperature which is above the melting point of the inventive ethylene copolymer wax(es) by at least 30° C.

If use is made of two or more different inventive ethylene copolymer waxes, the material is heated to a temperature which is above the melting point of the inventive ethylene copolymer wax which melts at the highest temperature. If use is made of two or more different inventive ethylene copolymer waxes, it is advantageous to heat the material to a temperature which is at least 10° C. above the melting point of the ethylene copolymer wax which melts at the highest temperature. If use is made of two or more different ethylene copolymer waxes, it is particularly advantageous to heat the material to a temperature which is at least 30° C. above the melting point of the ethylene copolymer wax which melts at the highest temperature.

In one embodiment of the present invention, the amount of Brønsted acid or Brønsted acids added is such that at least half, preferably at least 60 mol %, of the amino groups of the inventive ethylene copolymer wax(es) is/are protonated.

In one embodiment of the present invention, the amount of Brønsted acid or Brønsted acids added is such that the amino groups of the inventive ethylene copolymer wax(es) are quantitatively protonated.

In another embodiment of the present invention, the amount of Brønsted acid or Brønsted acids added may be more than necessary for the complete protonation of the amino groups of the inventive ethylene copolymer wax(es), for example an excess of up to 100 mol %, preferably up to 50 mol %.

The present invention also provides ionic ethylene copolymer waxes, also called inventive quaternized ethylene copolymer waxes below, obtainable via reaction of at least one inventive ethylene copolymer wax with an alkylating agent R¹¹-Z, where R¹¹ is selected from benzyl and C₁-C₁₀-alkyl, and in particular benzyl and methyl, and Z is selected from halogen, preferably chlorine, bromine or iodine, and R¹¹SO₄.

The present invention also provides a process for preparing inventive quaternized ethylene copolymer waxes obtainable via reaction of at least one inventive ethylene copolymer wax with an alkylating agent R¹¹-Z.

The amino groups present in the inventive ethylene copolymer waxes are partially or completely alkylated (quaternized) via the reaction of inventive ethylene copolymer waxes.

One or more inventive ethylene copolymer waxes is/are used as starting material for carrying out the inventive process for preparing inventive quaternized ethylene copolymer waxes. This or these is/are placed in a vessel, such as a flask, an autoclave or a tank, and the ethylene copolymer wax(es) and one or more Brønsted acids and, if appropriate, other substances, such as water and base, are heated, the sequence of addition of alkylating agent and also if appropriate other substances being as desired. If it is desired to prepare ionic ethylene copolymer waxes at a temperature above 100° C., it is advantageous to operate under increased pressure and to select the vessel appropriately. The resultant emulsion is homogenized, for example via mechanical or pneumatic stirring or by shaking. It is advantageous to heat the material to a temperature above the melting point of the inventive ethylene copolymer wax(es). It is advantageous to heat the material to a temperature which is above the melting point of the inventive ethylene copolymer wax(es) by at least 10° C., and it is particularly advantageous to heat the material to a temperature which is above the melting point of the inventive ethylene copolymer wax(es) by at least 30° C.

If use is made of two or more different inventive ethylene copolymer waxes, the material is heated to a temperature which is above the melting point of the inventive ethylene copolymer wax which melts at the highest temperature. If use is made of two or more different inventive ethylene copolymer waxes, it is advantageous to heat the material to a temperature which is at least 10° C. above the melting point of the ethylene copolymer wax which melts at the highest temperature. If use is made of two or more different ethylene copolymer waxes, it is particularly advantageous to heat the material to a temperature which is at least 30° C. above the melting point of the ethylene copolymer wax which melts at the highest temperature.

In one embodiment of the present invention, an excess of base, selected from aqueous sodium hydroxide solution and aqueous potassium hydroxide solution, is added, an excess meaning an excess based on equivalents of alkylating agent.

In one embodiment of the present invention, the amount of alkylating agent or alkylating agents added is such that at least half, preferably at least 60 mol %, of the amino groups of the inventive ethylene copolymer wax(es) are alkylated.

In one embodiment of the present invention, the amount of alkylating agent or alkylating agents added is such that the amino groups of the inventive ethylene copolymer wax(es) are quantitatively alkylated.

In another embodiment of the present invention, the amount of alkylating agent added may be an excess, based on amino groups of the inventive ethylene copolymer wax(es), for example an excess of up to 100 mol %, preferably up to 50 mol %.

The inventive process for preparing inventive quatemized ethylene copolymer waxes gives dispersions, preferably aqueous dispersions, which comprise inventive quaternized ethylene copolymer wax and are likewise provided by the present invention.

The inventive process for preparing ionic ethylene copolymer waxes gives dispersions, preferably aqueous dispersions, which comprise ionic ethylene copolymer wax and are likewise provided by the present invention.

Following the reaction of inventive ethylene copolymer wax with Brønsted acid or Brønsted acids it is also possible to carry out work-up steps, and by way of example the inventive dispersions obtainable may be filtered.

Inventive dispersions of inventive ionic ethylene copolymer waxes preferably have pHs of from 1 to 6.5, particularly preferably from 1.5 to 5.

Inventive dispersions of inventive quaternized ethylene copolymer waxes preferably have pHs of from 7 to 10, preferably from 8 to 9.5.

The solids content of inventive aqueous dispersions may be selected within a wide range. By way of example, suitable solids contents are from 0.1 to 50% by weight of ionic ethylene copolymer wax. The solids content of ionic ethylene copolymer wax may easily be adjusted by the person skilled in the art during the inventive process for preparing ionic ethylene copolymer wax via appropriate selection of the quantitative proportions of ethylene copolymer wax in relation to Brønsted acid or Brønsted acids.

The present invention also provides the use of inventive ethylene copolymer wax and the use of inventive ionic ethylene copolymer wax as microbicidal agent or as constituent of microbicidal agents.

The present invention also provides a method for the control of microorganisms, using inventive ethylene copolymer waxes or inventive ionic ethylene copolymer waxes.

To carry out the inventive method for controlling microorganisms, inventive ethylene copolymer waxes or inventive ionic ethylene copolymer waxes, for example in the form of their aqueous solution or dispersion, may be placed on a substrate which has been infested by microorganisms.

An example of a procedure for carrying out the inventive method for controlling microorganisms places inventive ethylene copolymer waxes or inventive ionic ethylene copolymer waxes, by way of example in the form of their aqueous solution or dispersion, in a solution, suspension or emulsion which has been infested by microorganisms.

Examples of suitable substrates which may be infested by microorganisms are textiles, in particular textiles for the care of the sick and for the genital area. Other substrates which may be mentioned by way of example are surfaces of furniture and of equipment, and also floors and walls, in particular in toilets, in bathrooms, in swimming pools, in hospitals, and within hospitals in particular in spaces used for intensive care and for the care of babies. Facades and roofs are also suitable. Other suitable substrates are foams.

In one embodiment of the present invention, inventive ethylene copolymer wax or inventive ionic copolymer wax is mixed into paints, such as emulsion paints, or into formulations for renders.

The inventive ethylene copolymer waxes and the inventive ionic ethylene copolymer waxes may also be used for prophylactic treatment to control microorganisms. The present invention also provides a method for the prophylactic treatment of substrates via treatment with inventive ethylene copolymer waxes or with inventive ionic ethylene copolymer waxes, substrates here being as defined above.

It is also possible to incorporate inventive ethylene copolymer waxes, inventive ionic ethylene copolymer waxes, and inventive quaternized ethylene copolymer waxes into polymers, for example via blending or coextrusion. Microbicidal polymers are obtained.

The invention is illustrated via examples.

### EXAMPLES

### Preparation of ethylene copolymer wax

### Ethylene and N,N-dimethylaminoethyl acrylate

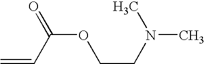

were copolymerized continuously in a high-pressure autoclave described in the literature (M. Buback et al., Chem. Ing. Tech. 1994, 66, 510). To this end, ethylene (12.0 kg/h) was continuously fed at the reaction pressure of 1700 bar into the high-pressure autoclave. Separately from this, the amount stated in table 1 of N,N-dimethyl-aminoethyl acrylate, if appropriate diluted with the amount stated in column 5 of table 1 of isododecane, was compressed first to an intermediate pressure of 260 bar and then, with the aid of another compressor, continuously fed at the reaction pressure of 1700 bar into the high-pressure autoclave. Separately from this, the amount stated in table 1 of initiator solution, composed of tert-butyl peroxypivalate (in isododecane, concentration see table 1) was continuously fed at the reaction pressure of 1700 bar into the high-pressure autoclave. Separately from this, if appropriate, the amount stated in table 1 of propionaldehyde was first compressed to an intermediate pressure of 260 bar and then, with the aid of another compressor, continuously fed at the reaction pressure of 1700 bar into the high-pressure autoclave. The reaction temperature was about 220° C. This gave inventive ethylene copolymer wax with the analytical data seen in table 2.

TABLE 1
Preparation of inventive ethylene copolymer waxes

| | | | | | | PO | | | ECW |
|---|---|---|---|---|---|---|---|---|---|
| | T_{reactor} | Ethylene | DMA3 | | PA | in ID | | | discharged |
| No. | [° C.] | [kg/h] | [g/h] | ID | [ml/h] | [l/h] | c(PO) | Conversion | [kg/h] |
| 1.1 | 220 | 12 | 234 | 526 | 280 | 1.4 | 0.02 | 20 | 2.5 |
| 1.2 | 221 | 12 | 468 | 292 | 330 | 1.0 | 0.04 | 18 | 2.5 |
| 1.3 | 220 | 12 | 945 | — | 190 | 1.8 | 0.05 | 19 | 3.0 |
| 1.4 | 220 | 12 | 1348 | — | 100 | 2.27 | 0.07 | 22 | 3.6 |
| 1.5 | 219 | 12 | 2387 | — | — | 3.53 | 0.12 | 19 | 4.2 |
| 1.6 | 220 | 12 | 1376 | — | — | 2.47 | 0.09 | 21 | 3.6 |

T_{reactor} is the maximum internal temperature of the high-pressure autoclave.

Abbreviations: DMA3: N,N-dimethylaminoethyl acrylate, PA: propionaldehyde, ID: isododecane (2,2,4,6,6-pentamethylheptane), PA in ID: solution of propionaldehyde in isododecane, total volume of solution.

PO: tert-butyl peroxypivalate, ECW: ethylene copolymer wax

c(PO): concentration of PO in ID in moll

The conversion is based on ethylene and is stated in % by weight

TABLE 2
Analytical data for the inventive ethylene copolymer waxes

| | | | Content | | | | |
|---|---|---|---|---|---|---|---|
| | Content of | Content of | of | Content | | | |
| | ethylene [% | DMA3 [% | ethylene | of DMA3 | ν | Tₘₑₗₜ | |
| No. | by weight] | by weight] | [mol %] | [mol %] | [mm²/s] | [° C.] | ρ [g/cm³] |
| 1.1 | 94.2 | 5.8 | 98.8 | 1.2 | 1260 | 103.7 | 0.9285 |
| 1.2 | 86.0 | 14.0 | 96.9 | 3.1 | 1190 | 97.3 | 0.9294 |
| 1.3 | 76.4 | 23.6 | 94.3 | 5.7 | 1130 | 86.1 | 0.9288 |
| 1.4 | 72.9 | 27.1 | 93.2 | 6.8 | 1160 | 77.8 | 0.9295 |
| 1.5 | 55.7 | 44.3 | 86.5 | 13.5 | 930 | 65.9 | n.d. |
| 1.6 | 68.4 | 31.6 | 91.7 | 8.3 | 2720 | 76.7 | 0.9313 |

n.d.: not determined.
“Content” is the proportion of copolymerized ethylene or DMA3 in the respective ethylene copolymer wax.
ν: dynamic melt viscosity, measured at 120° C. to DIN 51562.

The content of ethylene and N,N-dimethylaminoethyl acrylate in the inventive ethylene copolymer waxes was determined via ¹H NMR spectroscopy.

The density was determined to DIN 53479. The melting point Tₘₑₗₜ or melting range was determined by DSC (Differential scanning calorimetry) to DIN 51007.

### Preparation of Inventive Dispersions of ionic ethylene copolymer waxes

The amount stated in table 3 of ethylene copolymer wax of example 1 formed an initial charge in a 2-liter autoclave with anchor stirrer. The material was heated to 140° C., with stirring, and then the amount stated in table 3 of 37% strength by weight aqueous hydrochloric acid was added dropwise within a period of 30 minutes. Stirring was then continued for a further hour at 99° C., and then the material was cooled to room temperature within a period of 15 minutes.

Filtration with a Perlon filter (100 μm) gave the inventive dispersions 2.1 to 2.4.

TABLE 3
Inventive dispersions of ionic ethylene copolymer waxes

| | | | Amount | Amount | Molar ratio of | | |
|---|---|---|---|---|---|---|---|
| | ECW | Amount of | of HCl | of H₂O | amino groups: | pH of | Solids content |
| No. | No. | ECW [g] | [g] | [ml] | HCl | dispersion | [% by weight] |
| 2.3.1 | 1.3 | 200 | 30 | 1660 | 1:1 | 1.9 | 10.3 |
| 2.3.2 | 1.3 | 100 | 11.7 | 388.3 | 1:0.8 | 4.6 | 20.2 |
| 2.5 | 1.5 | 200 | 55.7 | 744 | 1:1 | 3.3 | 21.8 |
| 2.6 | 1.6 | 200 | 40 | 3760 | 1:1 | 2.9 | 4.7 |

Control of microorganisms

The procedure used to study effectiveness in controlling microorganisms was as follows.

50 ml of 1 M KH₂PO₄ were brought to pH 7.0, using KOH, and treated with 50 ml of the inventive dispersion 2.5 of table 3. This gave buffered dispersion 3.5, which was cloudy.

3.1. Study of Escherichia coli on Meat Peptone Agar Plates

Escherichia coli was prepared in the form of a suspension in 0.9% strength by weight aqueous NaCl, and plated out to give a dense lawn on meat peptone agar plates. Filter discs (Whatman Chromatography Paper, Cat. No. 3030 931) which had been stamped out with a hole punch and then dried in an autoclave for 20 min at 121° C. and 1.5 bar were then placed on the agar plates.

10 μl of buffered dispersion 3.5 at various dilutions (from 1:2 to 1:64, diluted with deionized water) were dribbled onto the filter discs pretreated as described above.

The meat peptone agar plates treated with Escherichia coli and buffered dispersion 3.5 were then incubated at 37° C. After 2 days of incubation at 37° C. there was a visible bacterial lawn over almost the entire agar plate. Extending as far as a dilution of 1:20, there was a discernible inhibitory zone of about 2 mm around the filter discs impregnated with buffered dispersion 3.5. No correlation was observed between concentration and inhibitory zone size.

3.2. Studies Carried Out on Liquids Comprising Microorganisms

The following nutrient solutions were used:

3.2.1. Meat Peptone,

Difco “Nutrient Broth” in the form of a powder composed of beef extract and peptone in a ratio of 3:5 by weight was diluted to 8 g/l with deionized water.

3.2.2. M12 Minimal Medium

The following were dissolved in 966 ml of doubly distilled water:

1 g of (NH₄)₂SO₄, 0.1 g of NaCl, 0.2 g of MgSO₄.7 H₂O, 0.02 g of CaCl₂.2 H₂O, 0.05 g of yeast extract,

and 1 ml of solution of trace elements, the content of the solution of trace elements being:

200 mg/l of FeSO₄.H₂O, 10 mg/l of ZnSO₄.7 H₂O, 3 mg/l of MnCl₂.4 H₂O, 30 mg/l of H₃BO₃, 20 mg/l of CoCl₂.6 H₂O, 1 mg/l of CuCl₂.6 H₂O, 2 mg/l of NiCl₂.6 H₂O, 3 mg/l of Na₂MoO₄.2 H₂O,

500 mg/l of Titriplex III (disodium salt of ethylenediaminetetraacetic acid in the form of dihydrate)

The resultant solution was autoclaved for 20 minutes in an autoclave at 121° C. and 1.5 bar and then mixed with

20 ml of 10% strength by weight KH₂PO₄— solution

10 ml of vitamin stock solution and

4 ml of 50% strength by weight aqueous glucose solution.

The vitamin stock solution was a sterile-filtered aqueous solution of 200 mg/l of biotin, 200 mg/l of folic acid, 20 mg/l of p-aminobenzoic acid, 20 mg/l of riboflavin, 40 mg/l of calcium pantothenate, 140 mg/l of nicotinic acid, 40 mg/l of pyridoxine-HCl, 200 mg/l of myoinositol, 40 mg/l of thiaminium-HCl.

The buffered dispersion 3.5 described above was diluted with water to various concentrations (in the range from 2.5% by volume of buffered dispersion 3.5 to 0.001 % by volume of buffered dispersion 3.5).

The nutrient solutions 3.2.1 and 3.2.2 were inoculated in separate experiments with the microorganisms Escherichia coli, Bacillus thuringiensis and Pseudomonas putida, and treated with buffered dispersion 3.5 at different concentrations. To give a blind specimen, in each case a 10% by weight KH₂PO₄ solution not diluted with buffered dispersion 3.5 to give nutrient solutions was also added to 3.2.1 or 3.2.2.

For liquid cultures without buffered dispersion, rapid bacterial growth was observed, indicated via marked clouding of the medium within 48 hours. Starting at a certain required minimum concentration of buffered dispersion, no growth was then observed in liquid cultures (see table 4). The clouding was very visible.

The required minimum concentration varied, depending on the type of bacterium. The results are given in table 4.

TABLE 4
Control of microorganisms using buffered dispersion 3.5

| | | | Minimum |
|---|---|---|---|
| | | | concentration of |
| Experiment | | | buffered dispersion |
| No. | Strain | Medium | 3.5 [% by volume] |
| 3.1 | Escherichia coli | Meat peptone | 0.025 |
| 3.2 | Escherichia coli | Mineral salt | 0.025 |
| | | (M12) | |
| 3.3 | Bacillus | Meat peptone | 0.25 |
| | thuringiensis | | |
| 3.4 | Bacillus | Mineral salt | 0.25 |
| | thuringiensis | (M12) | |
| 3.5 | Pseudomonas putida | Meat peptone | 0.25 |
| 3.6 | Pseudomonas putida | Mineral salt | 0.1 |
| | | (M12) | |

3.3. Comparison of Inventive Wax with Comparative Polymer from DE 101 27 513 A1

Example 2 from DE 101 27 513 was repeated. This gives comparative polymer C1. 1 g of comparative polymer C1 was then dissolved in 1 g of ethanol to give an ethanolic solution of comparative polymer C1.

218 μl of ethanolic solution of comparative polymer C1 were diluted with 782 μl of deionized water. 500 μl of the resultant aqueous solution of comparative polymer C1 were removed and diluted with 500 ml of 1.0 M KH₂PO₄ solution (pH 7.0). This gave a buffered solution of comparative polymer C1.

Diluted test solutions of C1 were prepared from buffered dispersion 3.5 and from buffered solution of comparative polymer C1, in each case using 1.0 M KH₂PO₄ solution (pH 7.0), and specifically in the range from 1:10 to 1:200 (in each case based on volume, so that, for example, a test solution of C1 diluted to 1:10 comprised one part by volume of buffered C1 comparative polymer solution and 9 parts by volume of 1.0 M KH₂PO₄-solution).

MP agar plates were inoculated with 50 μl of dense bacterial suspension (Escherichia coli XJS794) over the entire surface, with the aid of a Drigalski spatula. 10 μl of a test solution of C1 were then pipetted onto each of the agar plates.

As control, 10 μl of a 1.0 M KH₂PO₄ solution were pipetted onto another MP agar plate inoculated as described above (blind specimen).

The MP agar plates, inoculated and treated with test solution or with 1.0 M KH₂PO₄ solution, were then incubated at 37° C.

Inhibition of bacterial growth was then studied visually.

TABLE 5
comparison of inventive dispersion 3.5 and
buffered comparative C1 polymer solution

| | | Buffered comparative C1 |
|---|---|---|
| Dilution | Buffered dispersion 3.5 | polymer solution |
| 1:10 | Inhibition of | Inhibition of bacterial growth |
| | bacterial growth | |
| 1:20 | Inhibition of | Inhibition of bacterial growth |
| | bacterial growth | |
| 1:30 | Inhibition of | Inhibition of bacterial growth |
| | bacterial growth | |
| 1:40 | Inhibition of | Inhibition of bacterial growth |
| | bacterial growth | |
| 1:50 | Inhibition of | Inhibition of bacterial growth |
| | bacterial growth | |
| 1:60 | Inhibition of | No inhibition of bacterial |
| | bacterial growth | growth |
| 1:100 | Inhibition of | No inhibition of bacterial |
| | bacterial growth | growth |
| 1:200 | Inhibition of | No inhibition of bacterial |
| | bacterial growth | growth |

1. An ethylene copolymer wax with a molecular weight M_{w} in the range from 1000 to 20 000 g/mol, which comprises, as copolymerized comonomers,
(a) from 50 to 95% by weight of ethylene,
(b) from 5 to 50% by weight of at least one comonomer of the general formula I, 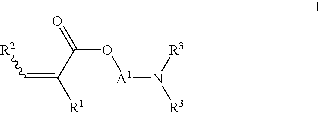
where the definition of the variables is as follows:
R¹ selected from hydrogen, unbranched and branched C₁-C₁₀-alkyl,
R² selected from hydrogen, unbranched and branched C₁-C₁₀-alkyl,
R³ identical or different and selected from hydrogen and unbranched and branched C₁-C₁₀-alkyl and C₃-C₁₂-cycloalkyl, where two radicals R³ may have bonding to one another to form a 3- to 10-membered ring,
A¹ a divalent group selected from C₁-C₁₀-alkylene, C₄-C₁₀-cycloalkylene and phenylene,
(c) from 0 to 30% by weight of other comonomers selected from isobutene and alkyl (meth)acrylate,
where all % by weight data are based on the entire weight of ethylene copolymer wax.

2. The ethylene copolymer wax according to claim 1, wherein the definition of the variables is as follows:
R¹ hydrogen or methyl,
R² hydrogen,
each R³ identical and selected from methyl and ethyl.

3. The ethylene copolymer wax according to claim 1, wherein (c) (meth)acrylic ester comprises C₁-C₄-alkyl (meth)acrylate.

4. A process for preparing ethylene copolymer waxes according to claim 1, which comprises copolymerizing ethylene and at least one comonomer which has at least one alkylated or cycloalkylated amino group, each of these groups having bonding via a spacer to a polymerizable group, and also, if appropriate, at least one other comonomer at temperatures in the range from 170 to 300° C. and pressures in the range from 500 to 4000 bar.

5. The process according to claim 4, wherein at least one comonomer which has at least one alkylated or cycloalkylated amino group, each of these groups having bonding via a spacer to a polymerizable group, is a comonomer of the general formula I.

6. A process for preparing ethylene copolymer waxes according to claim 1, wherein wax obtainable via copolymerization of ethylene with at least one comonomer of the general formula IV 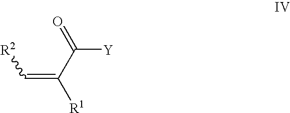
is reacted in a polymer-analogous reaction with at least one compound of the formula V 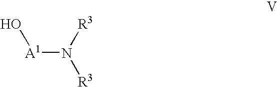
where
Y is selected from OH and O—R¹³ and
R¹³ is C₁-C₄-alkyl.

7. An ionic ethylene copolymer wax obtainable via reaction of at least one ethylene copolymer wax according to claim 1 with Brønsted acid.

8. An ionic ethylene copolymer wax obtainable via reaction of at least one ethylene copolymer wax according to claim 1 with an alkylating agent R¹¹-Z, where R¹¹ is selected from benzyl and C₁-C₁₀-alkyl and Z is selected from halogen and R¹¹SO₄.

9. A process for preparing ionic ethylene copolymer waxes according to claim 8, which comprises reacting an ethylene copolymer wax with a molecular weight M_{w} in the range from 1000 to 20 000 g/mol, which comprises, as copolymerized comonomers,
(a) from 50 to 95% by weight of ethylene,
(b) from 5 to 50% by weight of at least one comonomer of the general formula I, 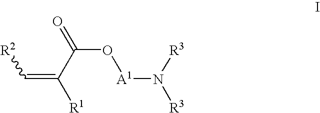
where the definition of the variables is as follows:
R¹ selected from hydrogen, unbranched and branched C₁-C₁₀-alkyl,
R² selected from hydrogen, unbranched and branched C₁-C₁₀-alkyl,
R³ identical or different and selected from hydrogen and unbranched and branched C₁-C₁₀-alkyl and C₃-C₁₂-cycloalkyl where two radicals R³ may have bonding to one another to form a 3- to 10-membered ring
A¹ a divalent group selected from C₁-C₁₀-alkylene, C₄-C₁₀-cycloalkylene and phenylene,
(c) from 0 to 30% by weight of other comonomers selected from isobutene and alkyl (meth)acrylate,
where all % by weight data are based on the entire weight of ethylene copolymer wax with an alkylating agent R¹¹-Z.

10. A process for preparing ionic ethylene copolymer waxes according to claim 8, which comprises reacting an ethylene copolymer wax with a molecular weight M_{w} in the range from 1000 to 20 000 g/mol which comprises, as copolymerized comonomers,
(a) from 50 to 95% by weight of ethylene,
(b) from 5 to 50% by weight of at least one comonomer of the general formula I, 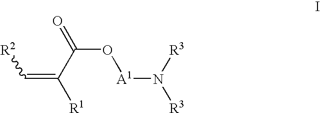
where the definition of the variables is as follows:
R¹ selected from hydrogen, unbranched and branched C₁-C₁₀-alkyl,
R² selected from hydrogen, unbranched and branched C₁-C₁₀-alkyl,
R³ identical or different and selected from hydrogen and unbranched and branched C₁-C₁₀-alkyl and C₁-C₁₂-cycloalkyl, where two radicals R³ may have bonding to one another to form a 3- to 10-membered ring,
A¹ a divalent group selected from C₁-C₁₀-alkylene, C₄-C₁₀-cycloalkylene and phenylene,
(c) from 0 to 30% by weight of other comonomers selected from isobutene and alkyl (meth)acrylate,
where all % by weight data are based on the entire weight of ethylene copolymer wax with a Brønsted acid.

11. An aqueous dispersion comprising ionic ethylene copolymer waxes according to claim 7.

12. The use of ethylene copolymer waxes according to claim 1 or of ionic ethylene copolymer waxes obtained from reacting said etheylene copolymer waxes with an alkylating agent R¹¹-Z, where R¹¹ is selected from benzyl and C₁-C₁₀-alkyl and Z is selected from halogen and R¹¹SO₄or a Brønsted acid as microbicidal agent or as constituent of microbicidal agents.

13. A method for the control of microorganisms, using ethylene copolymer waxes according to claim 1 or of ionic ethylene copolymer waxes obtained from reacting said etheylene copolymer waxes with an alkylating agent R¹¹-Z, where R¹¹ is selected from benzyl and C₁-C₁₀-alkyl and Z is selected from halogen and R¹¹SO₄or a Brønsted acid.

14. The method according to claim 13, wherein said ionic ethylene copolymer waxes are used in the form of aqueous dispersion.

15. A method for the prophylactic treatment of substrates to control microorganisms, which comprises treating them with ethylene copolymer waxes according to claim 1 or of ionic ethylene copolymer waxes obtained from reacting said etheylene copolymer waxes with an alkylating agent R¹¹-Z or a Brønsted acid.

16. The ethylene copolymer wax according to claim 2, wherein (c) (meth)acrylic ester comprises C₁-C₄-alkyl (meth)acrylate.

17. A process for preparing ethylene copolymer waxes according to claim 2, which comprises copolymerizing ethylene and at least one comonomer which has at least one alkylated or cycloalkylated amino group, each of these groups having bonding via a spacer to a polymerizable group, and also, if appropriate, at least one other comonomer at temperatures in the range from 170 to 300° C. and pressures in the range from 500 to 4000 bar.

18. A process for preparing ethylene copolymer waxes according to claim 3, which comprises copolymerizing ethylene and at least one comonomer which has at least one alkylated or cycloalkylated amino group, each of these groups having bonding via a spacer to a polymerizable group, and also, if appropriate, at least one other comonomer at temperatures in the range from 170 to 300° C. and pressures in the range from 500 to 4000 bar.

19. A process for preparing ethylene copolymer waxes according to claim, wherein wax obtainable via copolymerization of ethylene with at least one comonomer of the general formula IV 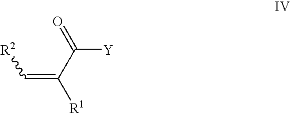
is reacted in a polymer-analogous reaction with at least one compound of the formula V 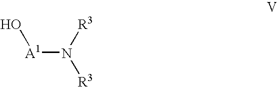
where
Y is selected from OH and O—R¹³ and
R¹³ is C₁-C₄-alkyl.

20. A process for preparing ethylene copolymer waxes according to claim 3, wherein wax obtainable via copolymerization of ethylene with at least one comonomer of the general formula IV 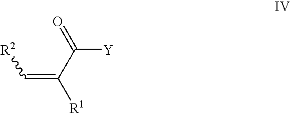
is reacted in a polymer-analogous reaction with at least one compound of the formula V 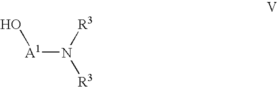
where
Y is selected from OH and O—R¹³ and
R¹³ is C₁-C₄-alkyl.
